(19) United States
(12) Patent Application Publication
(10) Pub. No.: US 2018/0185894 A1
(43) Pub. Date: Jul. 5, 2018

(54) BIOREMEDIATION OF HEAVY METAL CONTAMINATED GEOMATERIALS BY INDIGENOUS MICROORGANISMS

(71) Applicant: BioCement Technologies, Inc, Seattle, WA (US)

(72) Inventor: Malcolm B. Burbank, Pullman, WA (US)

(73) Assignee: BioCement Technologies, Inc, Seattle, WA (US)

(21) Appl. No.: 15/905,651

(22) Filed: Feb. 26, 2018

Related U.S. Application Data
(63) Continuation of application No. PCT/US2016/046694, filed on Aug. 12, 2016.
(60) Provisional application No. 62/210,533, filed on Aug. 27, 2015.

(51) Int. Cl. B09C 1/10; C12R 1/01; C12P 3/00

(52) U.S. Cl. CPC B09C 1/10; B09C 2101/00; C12P 3/00; C12R 1/01

(57) ABSTRACT

A method for increasing the concentration of metal carbonates in a heavy metal contaminated geomaterial utilizing indigenous ureolytic microorganisms. The method may be used for bioremediation of heavy metal contaminated geomaterials.

### FIELD OF THE INVENTION

This application pertains to the field of remediation of soils or other geomaterials that are contaminated with heavy metals.

### BACKGROUND OF THE INVENTION

Soils may become contaminated by the accumulation of heavy metals and metalloids through emissions from rapidly expanding industrial areas, mine tailings, disposal of high metal wastes, leaded gasoline and paints, land application of fertilizers, animal manures, sewage sludge, pesticides, wastewater irrigation, coal combustion residues, spillage of petrochemicals, and atmospheric deposition. Heavy metals may be cationic or anionic and constitute an ill-defined group of inorganic chemical hazards. Cationic metals most commonly found at contaminated sites are lead (Pb), chromium (Cr), arsenic (As), zinc (Zn), cadmium (Cd), copper (Cu), mercury (Hg), nickel (Ni), and manganese (Mn). Anionic metals, those that combine with oxygen and are negatively charged, most commonly found at contaminated sites are arsenic (As), molybdenum (Mo), selenium (Se), and boron (B).

Soils are the major sink for heavy metals released into the environment by anthropogenic activities and, unlike organic contaminants which are oxidized by microbial action, most metals do not undergo microbial or chemical degradation, and their total concentration in soils persists for a long time after their introduction.

The overall objective of any soil remediation approach is to create a final solution that is protective of human health and the environment. Several technologies currently exist for the remediation of metal contaminated soil, including isolation techniques whereby contaminated soil is capped or otherwise maintained in place to prevent contamination of adjacent soils, immobilization techniques whereby fixing amendments are added to contaminated soils in order to alter the soil metals to more geochemically stable phases, soil washing by means of physical and/or chemical procedures to extract metal contaminants from soils, and chemical treatment of soils to reduce the toxicity or mobility of heavy metals. Each of these techniques has disadvantages, including high expense, the need for construction of barriers to prevent migration of contaminants, the need to remove or disturb soil prior to treatment, and the need to constantly monitor the treated soils following treatment.

Phytoremediation, using metal-accumulating plants to remove heavy metals and first introduced about 30 years ago, is based on the fact that plants may remove and stabilize metal contaminants. Phytoremediation is an energy efficient, aesthetically pleasing method of remediating sites with low to-moderate levels of contamination, and it can be used in combination with other remedial methods as a finishing step to the remedial process.

The advantages of phytoremediation compared with classical remediation are that (i) it is more economically viable using the same tools and supplies as agriculture, (ii) it is less disruptive to the environment and does not involve waiting for new plant communities to recolonize the site, (iii) disposal sites are not needed, (iv) it is more likely to be accepted by the public as it is more aesthetically pleasing then traditional methods, (v) it avoids excavation and transport of polluted media thus reducing the risk of spreading the contamination, and (vi) it has the potential to treat sites polluted with more than one type of pollutant. However, there are several disadvantages of phytoremediation techniques including (i) it is dependent on the growing conditions required by the plant (i.e., climate, geology, altitude, and temperature), (ii) large-scale operations require access to agricultural equipment and knowledge, (iii) success is dependent on the tolerance of the plant to the pollutant, (iv) contaminants collected in senescing tissues may be released back into the environment in autumn, (v) contaminants may be collected in woody tissues used as fuel, (vi) the time taken to remediate sites far exceeds that of other technologies, and (vii) contaminant solubility may be increased leading to greater environmental damage and the possibility of leaching.

Recently, Kang et al, Ecological Engineering, 74:402-407 (2015) disclosed that soil contaminated with lead may be bioremediated by the introduction into the soil of cultured ureolytic bacteria which produce carbonates and precipitate lead carbonate (PbCO₃). Kang found that, following the culturing of the ureolytic bacteria and subsequent introduction of the cultured bacteria into the soil sample, divalent Pb concentrations in the soil samples tested were reduced by about 60%.

Crawford et al, U.S. Pat. No. 8,420,362, disclose that the concentration of calcium carbonate in soil may be increased by specifically encouraging the growth of ureolytic microorganisms that exist in the soil and adding a source of calcium. The ureolytic microorganisms convert urea to ammonium and carbonate ions and the carbonate ions combine with the added calcium to form calcium carbonate, which serves to cement soil particles together and harden the soil.

The method of Crawford requires the presence of sufficient ureolytic microorganisms in the soil and Crawford discloses that there are tests that exist that are useful to determine if a soil sample contains a microorganism that is capable of hydrolyzing urea.

Numerous authors have reported that the population of microorganisms is greatly reduced in soil samples that are contaminated with heavy metals. Kim, Marine Ecology, 26:203-206 (1985), reported that the number of bacteria present in subsurface waters is directly related to the level of metal contaminants present in the water. Oliveira, Journal of Bioscience and Bioengineering, 102(3):157-161 (2006), reported that the activity of the enzyme dehydrogenase, a sensitive assay for determining the effect of heavy metals on the physiologically active soil microbial biomass, was reduced by about 90% in a contaminated soil sample in related to a control soil uncontaminated sample.

As discussed in more detail below, the inventor tested soil samples that were contaminated with heavy metals and in which, prior to treatment of the soil as described below, no microbes were detected.

### DESCRIPTION OF THE DRAWING

FIG. 1 shows the spectrum of an X-ray diffraction study that was performed on untreated control soil. X-axis is 2-theta scale. Y-axis is intensity Lin (counts/second).

FIG. 2 shows the spectrum of an X-ray diffraction study that contains spikes indicating the presence of PbCO₃ (cerussite) in soil following treatment.

### DESCRIPTION OF THE INVENTION

It has been unexpectedly discovered that indigenous microorganisms can be utilized in heavy-metal contaminated geomaterial, such as soil and sediment, to precipitate the heavy metals in the form of metal carbonates. According to this method, the growth of ureolytic microorganisms is enhanced in situ, which microorganisms hydrolyze urea to ammonium and carbonate ions, and then which carbonate ions spontaneously bind with metals in the geomaterial to form metal carbonates. It has further been unexpectedly discovered that this method is useful, even in situations where no microorganisms, and particularly no ureolytic microorganisms, can be grown from soil on direct culture.

Thus, in one embodiment, the invention is a method for increasing the concentration of metal carbonates, other than calcium carbonate, in a heavy metal contaminated geomaterial. According to this embodiment of the invention, the concentration of metal carbonates is increased by enhancing the growth of ureolytic microorganisms within a heavy metal contaminated geomaterial by an enrichment process of providing a source of nutrients and urea and allowing the ureolytic microorganisms to convert the urea to ammonium and carbonate ions, which carbonate ions then combine with heavy metal ions within the contaminated geomaterial to form metal carbonates.

For purposes of this application, a heavy-metal contaminated geomaterial sample is one that contains one or more metals at a level that equals or exceeds the “intervention value” of the applicable Dutch Standards, which corresponds to the “action level,” the term used by the U.S. Environmental Protection Agency (EPA).

Table 1 shows the Target and Intervention Values (Action Levels) for metals in soil as published in the Dutch Standard in 2009.

TABLE 1

| | | Intervention |
|---|---|---|
| | Target Value | Value Soil |
| Metal | (μg/l) | (mg/kg) |
| Antimony | 0.15 | 22 |
| Arsenic | 7.2 | 76 |
| Barium | 200 | |
| Cadmium | 0.06 | 13 |
| Chromium | 2.5 | |
| Chromium III | | 180 |
| Chromium VI | | 78 |
| Cobalt | 0.7 | 190 |
| Copper | 1.3 | 190 |
| Mercury | 0.01 | |
| Mercury (inorganic) | | 36 |
| Mercury (organic) | | 4 |
| Lead | 1.7 | 530 |
| Molybdenum | 3.6 | 190 |
| Nickel | 2.1 | 100 |
| Zinc | 24 | 720 |
| Beryllium | 0.05 | 30 |
| Selenium | 0.07 | 100 |
| Tellurium | | 600 |
| Thallium | 2 | 15 |
| Tin | 2.2 | 900 |
| Vanadium | 1.2 | 250 |
| Silver | | 15 |

The method of this application may be used to increase the solubility of certain metals in a geomaterial for the purpose of “washing” the metal from contaminated geomaterial. Conversely, the method may be used to reduce the solubility of certain metals, such as to reduce the potential for the metals to leach from soil or liquid media into drinking water.

This process is a form of microbial induced carbonate precipitation (MICP) and, according to the present application, differs from prior art MICP methods in that the majority, and preferably all, of the microorganisms that are involved in the MICP of this application are indigenous.

The term “geomaterial” means a geologic or geologically derived material, examples of which include soil and rock.

The term “indigenous” when referring to microorganisms means originating and living or occurring naturally in an area or environment and excludes microorganisms that have been exogenously added to the area or environment unless such exogenously added microorganisms had been added to the area or environment at a time sufficiently distant in the past to permit the added microorganisms to adapt to the area or environment. For purposes of this application, a microorganism is considered to be indigenous if it was added to a geomaterial at least one week ago. Likewise, a microorganism is considered to be exogenous if it was added to a geomaterial less than one week ago.

Although it is preferred that no exogenous microorganisms are added to a geomaterial when performing the present invention, the utilization of exogenous microorganisms in addition to performing the steps of the present method is considered to be within the scope of the present method, so long as the steps of the present method are performed.

The geomaterial utilized in the present method may be varied provided that it has a structure with interconnected pores or fractures and contains within it a population of microorganisms that are capable of hydrolyzing urea. For example, the geomaterial may be rock, typically sedimentary rock such as a terrigenous, chemical/biochemical or organic sedimentary rock. Examples of sedimentary rock that are suitable for the present method include conglomerate, breccia, sandstone, siltstone, shale, limestone, gypsum, dolostone, and lignite. As another example, either as an alternative to or in combination with rock, the geomaterial may be unconsolidated or partially consolidated porous medium such as soil (e.g. gravel, sand, silt, clay with or without organics such as peat) or sediments. The geomaterial of the present method may also be fractured igneous or metamorphic rock. Volcanic rock containing interconnected pores may also be utilized as the geomaterial of the present method.

It is not necessary to determine the identity of particular microorganisms that may be present in the geomaterial. However, it may be helpful to describe particular microorganisms that may be present. The microorganisms that are suitable for the method of the invention may constitutively express urease so that urease is expressed regardless of ammonia or nitrogen compound concentration. Such organisms include the following bacteria: Sporosarcina pasteurii, Sporosarcina ureae, and Pseudomonas aeruginosa. Other microorganisms that are suitable for the method of the invention include those in which urease is expressed only in the presence of urea. An example of a bacterium in which urease is expressed only in the presence of urea is Proteus vulgaris. Since there exist many bacteria that are able to hydrolyze urea in geomaterials that have never been isolated or characterized, the organisms listed here are meant to be examples. Many other known microbial genera and even previously unknown phylogenetic microbial groups present in geomaterials likely have the same capabilities for urea hydrolysis and are inherently included among the preferred indigenous microorganisms to be used in the present method.

If desired, a source of calcium ions may be added to the geomaterial in combination with the source of urea and nutrients. It is preferred that no source of calcium ions is added to the geomaterial to be treated. As disclosed in U.S. Pat. No. 8,420,362, the addition of urea, nutrients, and a source of calcium ions to a geomaterial will result in the formation of calcium carbonates, as ureolytic microorganisms will be preferentially promoted and will then hydrolyze urea to ammonia and carbonate ions, which carbonate ions will then combine with the calcium ions to form calcium carbonate. At sufficiently high concentrations, this production of calcium carbonate will result in cementation of a geomaterial. In addition, the presence of calcium ions, even at concentrations below that which will cause cementation, will compete with ions of heavy metal for carbonate formation.

Therefore, in order to avoid cementation and to minimize competition for carbonate ions by calcium, the method of the present application is preferably performed without any source of calcium ions being added. If a source of calcium ions is added, the amount of calcium ions added to the geomaterial should preferably be below that which will result in cementation of the geomaterial and most preferably should be below that which will provide a concentration of calcium ions of 10 mM or higher in the geomaterial to be treated. Most preferably, the amount of calcium ions that is added is insufficient to provide a concentration of calcium ions of 5 mM or higher. The addition of a liquid source that contains calcium ions at a concentration less than 5 ppm is considered to be trace and, therefore, not to be considered for purposes of this application as adding a source of calcium ions.

The source of nutrients that is utilized in the current method is any compound or combination of compounds that provides to microorganisms a source of energy and carbon, and preferably a source of trace minerals and vitamins. Examples of suitable nutrient sources include carbohydrates such as monosaccharides, disaccharides, oligosaccharides, and polysaccharides such as starch and cellulose; organic acids or their salts such as aliphatic, aromatic, and amino acids; casamino acids; hydrocarbons such as aliphatic and aromatic hydrocarbons; fatty acids or substituted acids such as keto-acids and hydroxy-acids; sugar alcohols such as glycerol and mannitol; multifunctional acids such as citrate; pyridines; purines; pyrimidines; biomass hydrolysate; molasses; yeast extract; corn steep liquor; peptones; tryptone; soytone; nutrient broth, and industrial waste stream products such as whey. A preferred nutrient source is molasses. A second preferred nutrient source is glycerin (glycerol). Another preferred nutrient source is acetate, such as sodium acetate. In a preferred embodiment, molasses and acetate, or molasses and glycerin, are utilized in combination as a nutrient source.

The urea may be provided in various forms. Preferably, the urea is provided as an aqueous solution in water.

The nutrients and urea may be added to the geomaterial in any manner by which these materials are made available to microorganisms. For example the nutrients and urea may be added under pressure, such as by flushing or injecting, such as in an aqueous solution, into or onto the geomaterial, or by spraying, dripping, or trickling onto or into the geomaterial.

In accordance with the present method, the nutrients and urea may be added simultaneously or sequentially. The concentration of the source of nutrients added to the geomaterial is that which is sufficient to encourage the growth of microorganisms within the geomaterial and will vary depending primarily on the particular source of nutrients that is added. It is conceived that if molasses is utilized as a source of nutrients, a preferred concentration of molasses is between about 0.005% to 0.05% by volume of the nutrient source. However, lower or higher concentrations of molasses may be added to a geomaterial so long as the concentration of molasses that is added is sufficient to encourage the growth of microorganisms with the material. Similarly, a preferred range of concentration of sodium acetate is 10 mM to 150 mM. However, as with molasses, lower or higher concentrations of sodium acetate may be utilized. If glycerin is utilized as the source of nutrients, a preferred concentration is 1.25 ml/L of 90+% glycerol, although concentrations higher or lower than this preferred concentration may be utilized, such as between 0.5 ml/L and 2.5 ml/L.

As stated above, urea may be added together with, or separately from, the nutrients. If urea was added at any time, then it may not be necessary to add additional urea during any subsequent treatment phase. The concentration of urea that is added to the geomaterial is that which is sufficient to produce sufficient carbonate to bind metal ions within the geomaterial. A preferred range of urea concentration that is added is between 250 mM to 2 M (2000 mM). Concentrations of urea lower than 250 mM, for example as low as 50 mM or even lower, may be utilized. However, the desired rise in pH and production of carbonate ions will be slowed. Concentrations of urea higher than 2 M may also be utilized. A preferred concentration of urea is between 250 to 1000 mM, with a most preferred range between 333 to 500 mM.

It is preferred, although not essential, that two or more iterations of enrichment by adding one or more of urea and a nutrient source are performed. It may be desirable to perform two to five, or even more, such as ten iterations of enrichment. More or less enrichment cycles may be utilized, depending on the initial numbers of indigenous bacteria present in the soil, the type of soil present, and the level and type of metal contamination. Additionally, it has been found that pH rises more rapidly with successive iterations, which is conceived to be due to the microbial population in a geomaterial becoming more and more exclusively composed of microorganisms that are ureolytic and that can survive at elevated pH. Further, with additional iterations, the formation of metal carbonates within the geomaterial is enhanced.

The presently disclosed method overcomes many disadvantages that are inherent to prior art MICP methods. The present method avoids problems due to clogging at the injection site associated with prior art methods that occurs due to the rapid production of metal carbonates when bacteria are injected with a source of urea. The present method also avoids the problem of uneven distribution of metal carbonate production within a geomaterial which likewise is due to the rapid production of carbonates at or near the site of injection. The problems with clogging and uneven carbonate production are related to the difficulties associated with uniform transport of bacteria and attachment of bacteria to soil surfaces. Many factors affect the transport of bacteria through, and attachment to, soil grains including the properties of the cell surface, ionic strength of the carrier solution, flow rate, van der Waals forces, and pore space geometry within the soil matrix. Additionally, because the present method does not require the growth of one or more selected exogenous microorganisms that must be protected within a geomaterial, there is no need to fix microorganisms to the geomaterial prior to combining the necessary reagents for the method. Another advantage of the present method is that a larger number of diverse urea-hydrolyzing microbial species may be utilized in the present method, in contrast to the methods of the prior art in which a finite number of microbial species are utilized. Therefore, the present method obviates the need to manipulate the environment to favor one or more particular microbial species. Also, because the microbial population utilized in the current method is indigenous, the microorganisms used in this method are adapted to the local environment and are not at a competitive disadvantage in relation to microorganisms that are already in the geomaterial.

In addition to overcoming the disadvantages inherent to prior art methods, the current method provides a simpler and more robust method for bioremediation of heavy metal contaminated geomaterials. The method may be practiced in any geomaterial, does not require the culturing of microorganisms, and does not require steps such as fixing microorganisms in the geomaterial prior to practicing the method.

Crawford, U.S. Pat. No. 8,420,362, describes a test to determine if a geomaterial contains a microorganism that is capable of hydrolyzing urea. In particular, the Crawford patent describes the Rapid Urease Test, also known as the CLO test (Campylobacter-like organism test), which is utilized in the medical field as a rapid test for diagnosis of Helicobacter pylori. The basis of the test is the ability of H. pylori to secrete the urease enzyme, which catalyzes the conversion of urea to ammonia and bicarbonate. The test is performed by placing a sample of a geomaterial into a medium containing urea and a pH sensitive indicator such as phenol red. If the sample contains urease, the urea in the medium will be converted to ammonia, which raises the pH of the medium and changes the color of the specimen from yellow (negative) to red (positive).

For purposes of the method for increasing the concentration of metal carbonates in a heavy metal contaminated geomaterial described in the present application, tests such as the Rapid Urease Test by themselves may be insufficiently sensitive due to the low concentrations of microbes that are typically present in heavy metal contaminated materials. Whereas a positive Rapid Urease Test establishes that a sample contains a sufficient concentration of ureolytic microorganisms for the method of this application, a negative Rapid Urease Test does not necessarily indicate that the method of the current application cannot be successfully implemented.

The present method requires the presence of indigenous ureolytic microorganisms in order to be successful. However, because contaminated soils are often severely depleted of microorganisms, standard methods of determining the presence of microorganisms in general, and ureolytic microorganisms in particular, may not be useful.

The inventor has determined that, even in samples from which no bacteria were able to be cultured, which would suggest that the sample is sterile, the method could successfully be utilized if the sample were treated, by addition of nutrients and urea, so as to specifically encourage the growth of ureolytic microorganisms within the sample. Following such treatment, culture of ureolytic microorganisms or tests such as the Rapid Urease Test may be positive. Even if the sample appears to be sterile following such treatment, further single or multiple rounds of treatment, with either or both of nutrients and urea, will often sufficiently stimulate the growth of ureolytic microorganisms within a material to be treated to allow such microorganisms to be detected in culture and/or to produce a positive Rapid Urease Test.

In geomaterials in which the presence of ureolytic microorganisms cannot be established, such as due to failure to grow in culture or production of a negative Rapid Urease Test, one or more rounds of supplementation with either or both of a source of nutrients and urea can be applied. It may be that only one round of supplementation may be necessary in order to obtain a positive culture or Rapid Urease Test. If, however, culture or other test for presence of urease positive microorganisms remains negative following a single round of supplementation, additional rounds of supplementation may be utilized, with each round utilizing either or both of urea and nutrients. Two rounds of supplementation, or three rounds or more may be needed. It is conceived that up to 20 rounds of supplementation may be necessary before one should concluded that the geomaterial to be treated contains insufficient numbers of ureolytic microorganisms so that the method of the present application is inapplicable.

The invention is further illustrated in the following non-limiting examples. All concentrations mentioned below are % w/w unless otherwise indicated.

### Example 1—Soil Samples

Heavy metal-contaminated soil was obtained using a backhoe from land adjacent to a zinc smelting plant at a Superfund Site in Government Gulch in Kellogg, Id. at a depth of 6 to 10 feet. The excavated soil was placed into several 5-gallon buckets, with each bucket containing about 50 to 75 pounds of soil. From this excavated soil, multiple replicates containing 30 grams of soil were prepared and these were loaded into nine (9) 30 cc syringe bodies that were being used as columns.

### Example 2—Treatment and Initial Enrichment of Samples

Three of the columns of Example 1 were labeled as controls. Three of the columns were labeled as Ca−, which indicated that no calcium would be utilized with this group of columns. Three of the columns were labeled as Ca+, which indicated that calcium would be utilized with this group of columns.

To each group of 3 columns, the appropriate treatment was applied. To the control columns, deionized water was added. To the Ca− group of columns, an enrichment solution containing 100 mM sodium acetate, 333 mM filtered sterile urea, 0.5 g/l corn steep liquid powder with 0.1% v/v of molasses was added. To the Ca+ group of columns, the enrichment solution as for the Ca− group was added, except that the enrichment solution for the Ca+ group further included 250 mM CaCl₂.

Three days after the enrichment treatment, the columns were drained and about 1 ml of the effluent from each column was collected in sterile centrifuge tubes. The effluent-containing tubes were centrifuged in order to collect any bacteria and particulates in the samples. The resulting pellet was washed and suspended twice in 1 ml of cold normal saline. A 30 μl aliquot from each of the columns was examined microscopically to visually detect the presence of planktonic bacteria.

Serial dilutions of 1:2, 1:20, and 1:100 were made of each collected sample and 50 μl of each dilution was placed on urea agar containing phenol red. This medium is an established medium for the detection of ureolytic bacteria, and non-ureolytic bacteria will grow in this medium as well. The inclusion of phenol red acts as a visual indicator of urea hydrolysis by ureolytic bacteria as it will turn red when urea is hydrolyzed and ammonium ions are released.

Visual microscopic examination of each of the aliquots failed to reveal the presence of bacteria from any of the columns. Additionally, none of the bacterial cultures produced colonies of bacterial growth.

### Example 3—Second Enrichment of Samples

The treatments of each of the groups of columns as described in Example 2 were repeated, followed by drainage and collection of effluent as described. The collected effluents were centrifuged as described and examined for the presence of planktonic bacteria. As in Example 2, visual microscopic examination of each of the aliquots failed to reveal the presence of bacteria from any of the columns. Additionally, serial dilutions of the effluent were plated as described in Example 2 and none of the bacterial cultures produced colonies of bacterial growth.

### Example 4—Third Enrichment of Samples

The treatments of each of the groups of columns as described in Examples 2 and 3 were repeated, followed by drainage and collection of effluent, and dilution and culture, as described. The collected effluents were centrifuged as described and examined for the presence of planktonic bacteria.

Following this third enrichment treatment, small numbers of planktonic bacteria were detected microscopically. Bacterial culture produced small amounts of colonies and, upon visual inspection of the cultured bacteria, it was noted that a large percentage of the isolated bacterial appeared to be malformed.

### Example 5—Fourth Enrichment of Samples

The treatments of each of the groups of columns as described in Examples 2 and 3 were repeated, followed by drainage and collection of effluent, and dilution and culture, as described. The collected effluents were centrifuged as described and examined for the presence of planktonic bacteria.

Following this fourth enrichment treatment, large numbers of planktonic bacteria were detected microscopically. Bacterial culture produced large amounts of colonies that were too numerous to count. Serial dilutions were performed in order to obtain a countable number of bacterial colonies.

Following this fourth round of enrichment, each of the columns was treated with two additional rounds of enrichment so that each soil sample received a total of six rounds of enrichment with nutrients and urea.

### Example 6—Analysis of Soil Sample

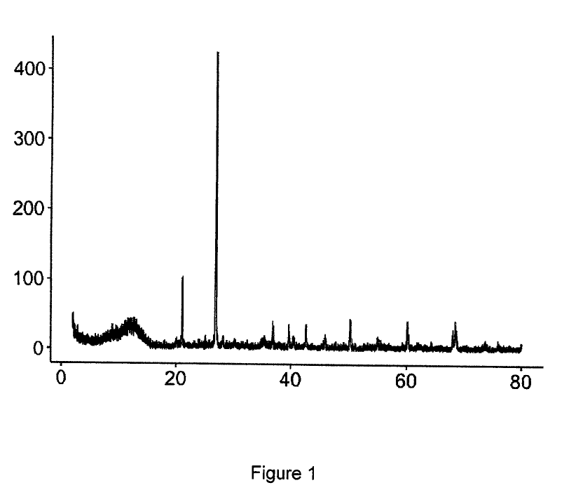

The level of heavy metal contaminants in the soil of Example 1 was determined by ICP-MS analysis of the soil in the control columns that were treated with deionized water only. Table 2 shows the levels of heavy metals in mg/kg for those metals that were found by ICP-MS analysis to be present at levels higher than the applicable Dutch Standard, as shown in Table 1. Additionally, an X-ray Diffraction study was performed on the control soil, the spectrum of which is shown in FIG. 1. As shown in FIG. 1, no metal carbonates were detected by X-ray Diffraction in control soil samples.

TABLE 2

| | Dutch Standard Intervention | Concentration of Metal |
|---|---|---|
| Metal | Value Soil (mg/kg) | (mg/kg) in Sample |
| Antimony | 22 | 110 |
| Arsenic | 76 | 84 |
| Cadmium | 13 | 37 |
| Copper | 190 | 320 |
| Lead | 530 | 20000 |
| Zinc | 720 | 5400 |

### Example 7—Formation of Metal Carbonates

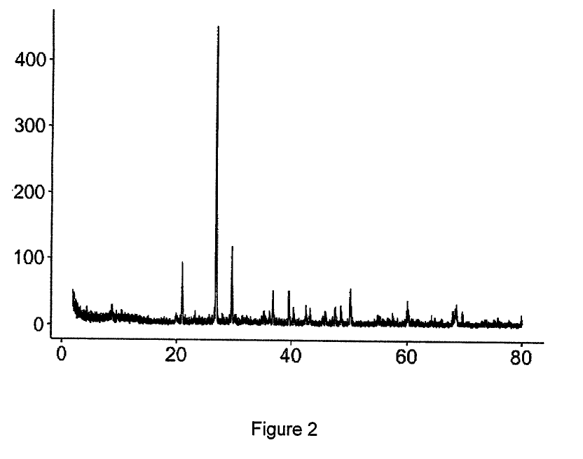

Following six rounds of enrichment, as discussed above in Example 4, soil samples were evaluated by X-ray diffraction for the presence of metal carbonates, particularly lead and zinc carbonates. The samples were removed from the columns and air dried prior to the X-ray diffraction analysis. The X-ray diffraction study revealed the presence of lead and zinc carbonates in the treated samples. FIG. 2 shows the X-ray Diffraction spectrum that contains spikes indicating the presence of PbCO₃ (cerussite).

### Example 8—Soil Samples

Heavy metal-contaminated soil samples were collected from the Government Gulch area of the Bunker Hill Mining and Metallurgical Complex Superfund site in Kellogg, Id. This superfund site is known to contain high concentrations of heavy metals, including lead, cadmium, zinc, and manganese.

The pH of the collected soil samples was determined to be 5.48. The concentrations of lead, cadmium, manganese, and zinc in the samples were determined and are shown below in Table 3.

TABLE 3
Concentration (μg/g)

| Pb | Cd | Mn | Zn |
|---|---|---|---|
| 20000 | 37 | 14000 | 5400 |

Sterile 60 ml syringe bodies were used as soil columns. Scour pad material was cut to fit the inside bottom of each column to minimize the loss of fines, and flexible rubber tubing was slipped over the needle end of the syringe body to clamp closed the draining end of the column. 30 grams of the contaminated soil that was relatively free of stones and plant material was added to each of the 6 test columns.

### Example 9—Treatment and Enrichment of Samples

The soil samples of Example 7 were treated in triplicate, with 10 ml of a sterile enrichment solution designed to enrich for ureolytic indigenous soil bacteria or with an equal volume of 10 mM CaCl₂ as a control. The first solution was stirred into the soil to ensure adequate wetting of the soil. Fresh sterile enrichment solution containing either 333 M urea, 0.5 g/L corn steep liquor and 50 mM sodium acetate or control solution containing 10 mM CaCl₂ was added to drained soil in columns every 4 days or when the pH in the test soil columns increased by more than 1 pH unit over a 24-hour period of time. A 1.0-point increase in pH over a 24-hour period is an early indication of urea hydrolysis. As the bacterial consortium becomes more prominently ureolytic, the rate of hydrolysis is increased and the pH increases more rapidly.

Approximately 1.8 ml of enrichment solution or CaCl₂ (pore fluid) was collected three times from each column, when the columns were drained to replace the enrichment or CaCl₂ solution. The collected pore fluid was centrifuged three times and suspended in 0.5 ml of sterile normal saline to collect the planktonic bacteria from the column for microscopic analysis and to collect bacteria for characterization. 200 μl of solution was reserved for serial dilution and plating as described below.

No bacteria were observed under magnification during the first enrichment of the enriched samples or at any stage in the control columns treated only with CaCl₂. A small number of bacteria were observed in the effluent of each of the columns that received enrichment solution after the second enrichment. No growth of bacteria was detected from the dilutions made after the first two enrichments or from any of the control columns. After the third enrichment, the number of bacteria in the soils that received the enrichment treatment appeared similar to the number of bacteria that we had previously observed in uncontaminated soils after a single enrichment. There was also an expected increase in the rate of urea hydrolysis as indicated by an increase in pH over time with each enrichment, most likely due to an increase in total ureolytic bacteria.

The pH in the enriched samples, containing approximately 20,000 ppm of Pb, slowly increased from 5.48 to 8.94 after receiving the 3 pulse injections over a 15-day time period.

After the final enrichment, all 6 of the columns (3 test columns and 3 control columns) were drained overnight. Triplicate test soil columns were treated with 10 ml of a solution containing 250 mM calcium chloride, 333 mM urea, 100 mM sodium acetate, 0.5 g/L glucose, and 0.5 g/L corn steep liquor with a final pH of 7. Triplicate control columns were treated only with 10 mM CaCl₂, pH 7. Each test soil column received a total of three injections of the solution or CaCl₂ (control) spaced three days apart.

### Example 10—Analytical Test of Soils and Results

Three treated and three controls columns of Example 8 were prepared for leaching as follows. Approximately 10 g of soil from each of three replicates (treated and control) was leached with a total of 300 ml of a leaching solution (10 mM CaCl₂, adjusted to pH 3 with 10⁻² mol/L of HNO₃) per sample over a three-week period and 10 ml of acidic CaCl₂. Prior to the first pulse injection of acidic CaCl₂, the soils were dried for 48 hours at 110° C. and then broken up with a metal rod to allow the leaching solution to infiltrate the soil. Then the acidified CaCl₂ was added to the soils by stirring the soil and acid solution until the soil in the columns was thoroughly wet with the solution.

The filtered, acidified leachate from each column was analyzed by ICP-MS to determine the metal concentrations that leached from the soil during the acid wash in the treated soil versus the untreated controls. The results showed that the leached metals in treated soils were reduced compared to metals leached from untreated soils. The range and mean of metals leached for the treated and untreated columns and the % reduction is presented in Table 4.

TABLE 4

| | Metal Eluting From Column (ug/L) | | | |
|---|---|---|---|---|
| | Pb | Cd | Mn | Zn |
| Treated range | 440- | 3.0- | 890- | 500- |
| [mean] | 2,000 | 15 | 2400 | 2500 |
| | [1,288] | [9.78] | [1878] | [1700] |
| Untreated Range | 1700- | 280- | 100,000- | 7,000- |
| [mean] | 2700 | 410 | 170,000 | 11,000 |
| | [2,200) | [343.3] | [135,000] | [8,833] |
| Mean % reduction | 41.5 | 97.2 | 98.6 | 80.8 |
| in solubility | | | | |

### Example 11—X-Ray Diffraction Studies

X-Ray diffraction (XRD) scans were performed on a Siemens D5000 theta-theta goniometer XRD equipped with a Cu X-ray tube and a solid-state (SiLI) wafer detector. Scans were performed at 40 kV and 30 mA tube power. Scan parameters: 2-theta range from 2 to 80 degrees at 0.02 step-size and 2 s step-time. The standard used to identify calcite in the samples was PDF 00-005-0586, a synthetic form of pure calcite. A focused scan over the 104 (hkl) calcite peak was performed using a step-time of 20 sec.

XRD scans showed the presence of calcite and PbCO₃ in the enriched soil samples. The presence of calcite in the treated samples was confirmed by X-ray diffraction (XRD). No calcite was detected in the control samples treated with 10 mM CaCl₂ alone. The results indicate precipitated calcite or other carbonates reduce the solubility of Pb, Cd, Mn and Zn in soils and that the precipitated metals are more resistant to solubilizing after exposure to acidic leaching solution.

The above examples show that metal carbonates are formed in heavy metal contaminated geomaterial from which bacteria were not detectable prior to enrichment with nutrients and urea by preferentially stimulating the growth of ureolytic bacteria by providing one or more rounds of enrichment with urea and nutrients and permitting the bacteria that have been preferentially stimulated to grow to hydrolyze urea to ammonium and carbonate ions, which carbonate ions spontaneously bind with metals in the geomaterial to form metal carbonates.

Various modifications of the above described invention will be evident to those skilled in the art. It is intended that such modifications are included within the scope of the following claims.

1. A method for increasing the concentration of metal carbonates other than calcium carbonates in a contaminated geomaterial, comprising:
adding a source of nutrients and urea to a geomaterial that contains indigenous microorganisms that are capable of hydrolyzing urea to ammonia and carbonate, thereby specifically promoting the growth of indigenous alkalinity-tolerant and ureolytic microorganisms within the geomaterial;
allowing the ureolytic microorganisms to produce carbonate ions; and
further allowing the carbonate ions to form metal carbonates within the geomaterials.

2. The method of claim 1 wherein the geomaterial is a heavy metal contaminated geomaterial.

3. The method of claim 1 comprising adding the source of nutrients substantially simultaneously with adding urea.

4. The method of claim 1 further comprising adding one or more of the nutrient sources and the urea plural times to the geomaterial.

5. The method of claim 1 wherein no exogenous microorganisms are added to the geomaterial.

6. The method of claim 1 wherein ureolytic microorganisms are not successfully cultured from the geomaterial prior to specifically promoting the growth of indigenous alkalinity-tolerant and ureolytic microorganisms within the geomaterial.

7. The method of claim 1, consisting essentially of promoting the growth of indigenous alkalinity-tolerant and ureolytic microorganisms within the geomaterial, allowing the ureolytic microorganisms to produce carbonate ions and further allowing the carbonate ions to form metal carbonates within the geomaterial.

8. The method of claim 1 wherein, if calcium is added to the geomaterial, the method comprises adding an amount of calcium to provide a calcium ion concentration of 10 mM or less in the geomaterial.

9. The method of claim 8 wherein the concentration of calcium ions is less than 5 mM in the geomaterial.

10. The method of claim 8 comprising adding less than 5 ppm calcium to the geomaterial.

11. The method of claim 8 wherein no calcium is added to the geomaterial.

12. A method for increasing the concentration of metal carbonates other than calcium carbonates in a contaminated geomaterial, the method consisting essentially of adding a source of nutrients and urea to a geomaterial that contains indigenous microorganisms that are capable of hydrolyzing urea to ammonia and carbonate, thereby specifically promoting the growth of indigenous alkalinity-tolerant and ureolytic microorganisms with the geomaterial.

13. The method of claim 1 wherein the source of nutrients is selected from a carbohydrate, a hydrocarbon, a sugar alcohol, an organic acid or a salt thereof, and combinations thereof.

14. The method of claim 13 wherein the source of nutrients is molasses, glycerin, sodium acetate, or combinations thereof.

15. The method of claim 1 wherein a concentration of the source of nutrients added is sufficient to promote the growth of indigenous alkalinity-tolerant and ureolytic microorganisms.

16. The method of claim 15 wherein a ratio of the concentration of the source of nutrients to that of the urea is 10 times or greater.

17. The method of claim 12 wherein the source of nutrients is selected from a carbohydrate, a hydrocarbon, a sugar alcohol, an organic acid or salt thereof, and combinations thereof.

18. The method of claim 17 wherein the source of nutrients is molasses, glycerin, sodium acetate, or combinations thereof.

19. The method of claim 12 wherein the source of nutrients is added substantially simultaneously with the addition of urea.

20. The method of claim 12 wherein the source of nutrients, the urea, or both, are added plural times to the geomaterial.
